(19) United States
(12) Patent Application Publication
(10) Pub. No.: US 2024/0142469 A1
(43) Pub. Date: May 2, 2024

(54) METABOLIC BIOMARKERS FOR ASSESSING HEART FAILURE IN A SINGLE VENTRICLE PEDIATRIC SUBJECT

(71) Applicant: The Trustees of Indiana University, Bloomington, IN (US)

(72) Inventors: Ronald Mark Payne, Bloomington, IN (US); Thomas Michael O'Connell, Bloomington, IN (US)

(21) Appl. No.: 18/546,910

(22) PCT Filed: Feb. 17, 2022
(86) PCT No.: PCT/US2022/016710

§ 371 Date: Aug. 17, 2023

Related U.S. Application Data
(60) Provisional application No. 63/150,786, filed on Feb. 18, 2021.

(51) Int. Cl. G01N 33/68; G01N 33/92

(52) U.S. Cl. CPC G01N 33/6893; G01N 33/92; G01N 2800/325; G01N 2800/52; G01N 2800/7076

(57) ABSTRACT

Methods to assess a pediatric subject having a single ventricle heart include identifying a difference between concentration levels of metabolic biomarkers and reference concentration levels and then assessing the pediatric subject based on the identified differences.

### CROSS-REFERENCE TO RELATED APPLICATION

This application claims priority to U.S. application Ser. No. 63/150,786, filed Feb. 18, 2021, the entire content of which is incorporated herein by reference.

### FIELD

The present disclosure relates to identification of pediatric patients having a single ventricle heart, and more particularly, to the use of metabolic biomarkers to assess single ventricle pediatric subjects for heart failure.

BACKGROUND

Pediatric subjects having a single ventricle (also referred to herein as “SV”) heart have a high rate of progression to heart failure. An ongoing, urgent need exists for rational based interventions before heart failure ensues. The presently disclosed subject matter addresses this need,

### SUMMARY

In one aspect, methods to assess a pediatric subject having a single ventricle heart include: determining a concentration level of one or more validated low molecular weight metabolic biomarkers in a biological sample from the subject; identifying a difference between the determined concentration level of the one or more metabolic biomarkers and a reference concentration level of the one or more metabolic biomarkers; and, assessing the pediatric subject based on the identified difference.

In another aspect; diagnostic kits for assessing an attribute of heart failure in a pediatric subject having a single ventricle heart, where the attribute of heart failure is associated with a low molecular weight metabolic biomarker profile, includes: a container with at least one validated low molecular weight Metabolic biomarker internal standard having a purity greater than 98.0%, a biological sample receiving vessel, and a sealing member configured to seal the sample receiving vessel after receiving the biological sample.

In another aspect, methods to identify a candidate low molecular weight metabolic biomarker that differentiates a pediatric, subject having a single ventricle heart without heart failure from a pediatric subject having a single ventricle heart with heart failure include obtaining a first biological sample from a pediatric subject having a single ventricle heart without heart failure and a second biological sample from a pediatric subject having a single ventricle heart with heart failure; determining a concentration level of one or more low molecular weight metabolic biomarkers in the first biological sample and the second biological sample; and, identifying one or more validated low molecular weight metabolic biomarkers that are present in a statistically significant, different level in the first and second biological samples, where the different level is characterized by an area under the receiver operator characteristic (ROC) curve (AUC) ranging from 0.60 to 1.00.

This summary is not intended to identify key or essential features of the claimed subject matter, nor is it intended to be used as an aid in limiting the scope of the claimed subject matter. Furthermore, it is envisioned that alternative embodiments may combine features of two or more of the above-summarized embodiments. Further embodiments, forms, features, and aspects of the present application shall become apparent from the description and figures provided herewith.

### BRIEF DESCRIPTION OF THE DRAWINGS

The concepts described herein are illustrative by way of example and not by way of limitation in the accompanying figures. For simplicity and clarity of illustration, elements illustrated in the figures are not necessarily drawn to scale. Where considered appropriate, reference labels have been repeated among the figures to indicate corresponding or analogous elements.

FIG. 1 illustrates a heatmap of 44 metabolites for which there was a significant difference in at least one of the inter-group comparisons. The metabolites were ordered using hierarchical cluster analysis.

FIG. 2 illustrates a Forest Plot of individual metabolite changes between groups with the log2-fold change plotted on the X-axis. (A) Control versus single ventricle (B) Control versus single ventricle heart failure and (C) Single ventricle versus single ventricle heart failure. Dot size indicates statistical significance: large dots p<0.05, medium dots=p<0.1, and small dots=p>0.1.

FIG. 3 illustrates receiver operating characteristic analyses of metabolite profiles.

FIG. 4 provides an overview of systemic derangements in SV patients lead to distinct metabolic patterns of heart failure.

### DETAILED DESCRIPTION

Before the present disclosure is further described, it is to be understood that this disclosure is not limited to particular embodiments described, as such may, of course, vary. It is also to be understood that the terminology used is for the purpose of describing particular embodiments only, and is not intended to be limiting, since the scope of the present disclosure will be limited only by the appended claims.

Unless defined otherwise, all technical and scientific terms have the same meaning as is commonly understood by one of ordinary skill in the art to which this disclosure belongs. All patents, applications, published applications and other publications are incorporated by reference in their entireties. If a definition set forth in this section is contrary to, or otherwise inconsistent with, a definition set forth in a patent, application, or other publication that is incorporated by reference, the definition set forth in this section prevails over the definition incorporated by reference.

The singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. The terms “including,” “containing,” and “comprising” are used in their open, non-limiting sense.

To provide a more concise description, some of the quantitative expressions are not qualified with the term “about.” It is understood that, whether the term “about” is used explicitly or not, every quantity is meant to refer to the actual given value, and it is also meant to refer to the approximation to such given value that would reasonably be inferred based on the ordinary skill in the art, including equivalents and approximations due to the experimental and/or measurement conditions for such given value.

Certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination. All combinations of the embodiments pertaining to the metabolic biomarkers represented by the variables are specifically embraced by the present disclosure just as if each and every combination was individually and explicitly disclosed. In addition, all subcombinations of the chemical groups listed in the embodiments describing such variables are also specifically embraced by the present disclosure just as if each and every such sub-combination of metabolic biomarkers was individually and explicitly disclosed.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. It should be further appreciated that although reference to a “preferred” component or feature may indicate the desirability of a particular component or feature with respect to an embodiment, the disclosure is not so limiting with respect to other embodiments, which may omit such a component or feature. Further, when a particular feature, structure, or characteristic is described in connection with an embodiment, it is submitted that it is within the knowledge of one skilled in the art to implement such feature, structure, or characteristic in connection with other embodiments whether or not explicitly described. Additionally, it should be appreciated that items included in a list in the form of “at least one of A, B, and C” can mean (A); (B); (C); (A and B); (B and C); (A and C); or (A, B, and C). Similarly, items listed in the form of “at least one of A, B, or C” can mean (A); (B); (C); (A and B); (B and C); (A and C); or (A, B, and C). Further, with respect to the claims, the use of words and phrases such as “a,” “an,” “at least one,” and/or “at least one portion” should not be interpreted so as to be limiting to only one such element unless specifically stated to the contrary, and the use of phrases such as “at least a portion” and/or “a portion” should be interpreted as encompassing both embodiments including only a portion of such element and embodiments including the entirety of such element unless specifically stated to the contrary.

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures unless indicated to the contrary. Additionally, the inclusion of a structural or method feature in a particular figure is not meant to imply that such feature is required in all embodiments and, in some embodiments, may not be included or may be combined with other features.

### Definitions

“Acylcarnitine” refers to fatty acyl esters of L-carnitine. “Carnitine” is an amino acid derivative and nutrient involved in lipid metabolism in mammals and other eukaryotes. Carnitine is in the chemical compound classes of P-hydroxyacids and quaternary ammonium compounds, and because of the hydroxyl-substituent, it exists in two stereoisomers: the biologically active enantiomer L-carnitine, and the essentially biologically inactive D-carnitine. Carnitine participates in the carnitine shuttle system which functions to transport fatty acids across the mitochondria(membrane for subsequent catabolism. Acylcarnitines (AC) are formed from a family of carnitine acyltransferases that exchange a CoA group for a carnitine. AcylCoA species cannot cross the mitochondrial membrane, but the ACs can. Once inside the mitochondria, these transferases can shuttle the ACs out of the mitochondria into the circulation. Serum ACs are thus a useful metabolic surrogate for intermediates along the β-oxidation pathway. Exemplary low molecular weight acylcarnitine metabolic biomarkers include, but are not limited to: carnitine (“C0”), acetylcarnitine (“C2”), propionycarnitine (“C3”), malonylcarnitine (“C3-DC”), hydroxybutyrylcarnitine (“C4-OH”), hydroxypropionylearnitine (“C3-OH”), propenoylcarnitine (“C:3:1”), butyrylcarnitine (“C4”), butenylcarnitine (“C4:1”), valerylcarnitine (“C5”), glutarylcarnitine (“C5-DC”), hydroxyhexanoylcarnitine (“C6-OH”), methylmalonylcarnitine (“C5-M-DC”), hydroxyvalerylcarnitine (“C5-OH”) leilethylmalonylcarnitine (“C3-DC-M”), tiglylcarnitine (“C5:1”) glutaconylcarnitine (“C5:1-DC”), hexanoylcarnitine (“C6”), fumarylcarnitine (“C4:1-DC”), hexenoylcarnitine (“C6:1”), pimeloylcarnitine (“C7-DC”), octanoylcarnitine (“C8”)), nonaylcarnitine (“C9”), decanoylcarnitine ((“C10”)), decenoylctirnitine (“C10:1”), decadienoylcarnitine (“C10:2”), dodecanoylcarnitine (“C12”), dodecanedioylcarnitine (“C12-DC'”), dodecenoylcarnitine (“C12:1”), tetradecanoylcarnitine (“C14”), tetradecenoylcarnitine (“C14:1”), hydroxytetradecenoylcarnitine (“C14:1-OH”), tetradecadienoylcarnitine ((“C14:2”)), hydroxytetradecanoylcarnitine (“C14:2-OH”)), hexadecanoylcanitine (“C16”), hydroxyhexadecanoylcarnitine (“C16-OH”), hexadecenoylcarnitine (“C16:1”), hhydroxyhexadecanoylcarnitine (“C16:1-OH”), hydroxydecadienoylcarnitine (“C16:2”), hydroxyhexadecanoylcarnitine (“C16:2-OH”), octadecanoylcarnitine (“C18”), octadecenoylcarnitine (“C18:1”), hydroxyoctadecenoylcarnitine (“C18:1-OH”), and octadecadienylcarnitine (“C18:2”).

“Area under curve” or “AUC” refers to area under a receiver operating characteristic (ROC) curve, AUC under a ROC curve is a measure of the predictive accuracy of a model. An area of 1 represents a perfect test, whereas an area of 0.5 represents a test that is no better than random guesses. In embodiments, the AUC may be at least approximately 0.700, at least approximately 0.750, at least approximately 0.800, at least approximately 0.850, at least approximately 0.900, or even at least approximately 0.950.

“Amino acid” refers to naturally occurring and non-natural synthetic amino acids, as well as amino acid analogs and amino acid mirnetics that function in a manner similar to the naturally occurring amino acids. There are 20 naturally occurring amino acids encoded by the genetic code. Amino acids can be referred to by either their commonly known three-letter symbols or by the one-letter symbols recommended by the IUPAC-IUB Biochemical Nomenclature Commission. Exemplary low molecular weight naturally occurring amino acid metabolic biomarkers include, but are not limited to, those provided in Table 1.

TABLE 1

Abbreviation

| Amino Acid | 3-Letter | 1-Letter |
|---|---|---|
| Alanine | Ala | A |
| Arginine | Arg | R |
| Leucine | Leu | L |
| Lysine | Lys | K |
| Asparagine | Asn | N |
| Aspartate | Asp | D |
| Methionine | Met | M |
| Phenylalanine | Phe | F |
| Cysteine | Cys | C |
| Glutamate | Glu | E |
| Proline | Pro | P |
| Serine | Ser | S |
| Glutamine | Gln | Q |
| Glycine | Gly | G |
| Threonine | Thr | T |
| Tryptophan | Trp | W |
| Histidine | His | H |
| Isoleucine | Ile | I |
| Tyrosine | Tyr | Y |
| Valine | Val | V |

Furthermore, there are a number of non-natural amino acids that meet the definition of an amino acid, possessing both an amino moiety and an acid moiety, but are not part of the set of 20 canonical amino acids. These non-natural amino acids have a wide range of functions including energy metabolism, cellular stress response, ammonia metabolism, and inflammatory and immune responses. Examples of low molecular weight non-natural amino acid metabolic biomarkers include, but are not limited to, 1-methylhistidine, 3-methylhistidine, 5-aminovaleric acid, α-aminoadipic acid, α-aminobutyric acid, β-aminobutyric acid, β-aminoisobutyric acid, γ-aminobutyric acid, acetylornithine, anserine, asymmetric dimethylarginine, betaine, carnosine, cis-4-hydroxyproline, citrulline, creatine, creatinine, cystine, dihydroxyphenylalanine, homoarginine, homoeysteine, kynurenine, methionine sulfoxide, iosine, ornithine, phenyluacetylglycine, phenylalanin betaine, proline betaine, sarcosine, symmetric dimethylargine, taurine, trans-4-hydroxyproline, and tryptophan betaine.

The term “organic acids” refers to compounds containing only an acid group. In some, cases, these are derived from amino acids through the action of deaminase transaminase enzymes, which remove the amino groups. In other eases, they are biosynthetic precursors to amino acids. Examples of organic acids are also found in the citric acid cycle which is one of the major pathways for cellular energy generation. Organic acids can also include compounds with more than one acid group, or chemical moieties including carbonyl and hydroxyl groups. Examples of low molecular weight organic acid metabolic biomarkers include, but are not limited to, those of Table 2.

TABLE 2

| Organic Acid | Chemical Class |
|---|---|
| Lactic acid | α-hydroxyacid |
| Malic acid | α-hydroxydicarboxylic acid |
| α-keto-β-methylvaleric acid | α-ketoacid |
| α-ketoisocaproic acid | α-ketoacid |
| α-ketoisovaleric acid | α-ketoacid |
| pyruvic acid | α-ketoacid |
| α-ketoglutaric acid | α-ketodicarboxylic acid |
| oxalacetic acid | α-ketodicarboxylic acid |
| acetic acid | carboxylic acid |
| butyric acid | carboxylic acid |
| propionic acid | carboxylic acid |
| fumaric acid | dicarboxylic acid |
| succinic acid | dicarboxylic acid |
| 3-indoleacetic acid | indole carboxylic acid |
| isocitric acid | hydroxytricarboxylic acid |
| citric acid | tricarboxylic acid |

“Assess” or “assessing” refers to both quantitative and qualitative determination in the sense of obtaining an absolute value for the amount or concentration of the metabolic biomarker or metabolic biomarkers to be analyzed present in the sample, and also to obtaining an index, ratio, percentage or other value indicative of the level of metabolic biomarker in the sample. Assessment may be direct or indirect, and the chemical species actually detected need not be the analyte itself but may, for example, be a derivative thereof. The purpose of such assessment of metabolic biomarkers may be different. In particular, an assessment may be performed for differentiating single ventricle pediatric subjects not having heart failure from single ventricle pediatric subjects having heart failure. The purpose of an assessment may also be determining a risk of developing heart failure and long-term outcome in a subject. Assessment also encompasses determining a stage in progression of the heart failure or the potential improvement in heart failure resulting from a therapeutic intervention. Assessment as used herein also encompasses determining a stage in progression to heart failure.

“Bile acid” refers to metabolites that promote fat absorption by acting as potent “digestive surfactants” to lipids (including fat-soluble vitamins) by acting as emulsifiers. Bile acids constitute a large family of molecules, composed of a steroid structure with four rings, a five or eight carbon side-chain terminating in a carboxylic acid, and the presence and orientation of different numbers of hydroxyl groups. The four rings are labeled from left to right on Formula 1, shown below as A, B, C, and D, with the D-ring being smaller by one carbon than the other three. The hydroxyl groups have a choice of being in 2 positions, beta (solid pie-shaped line), or alpha (dashed line). All bile acids have a hydroxyl group on position 3, which was derived from the parent molecule, cholesterol. In cholesterol, the 4 steroid rings are flat and the position of the 3-hydroxyl is beta.

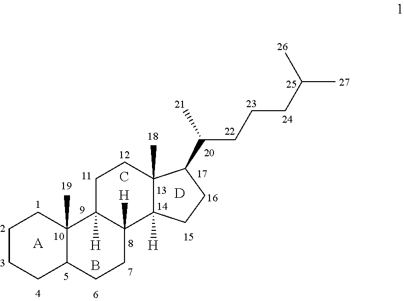

The immediate products of the bile acid synthetic pathways are referred to as primary bile acids. Cholic acid (Formula 2) and chenodeoxycholic acid )Formula 4) are two forms of primary bile acids formed in humans. The action of intestinal bacterial flora on primary bile acids results in the formation of secondary bile acid species: deoxycholic acid (Formula 6), lithocholic acid (Formula 8), and tirsodeoxycholic acid (Formula 10). Deoxycholic acid is derived from cholic acid and lithocholie acid and ursodeoxycholic acid are derived from chenodeoxycholic acid. “Bile acid,” also includes bile acid alcohols, sterols, and salts thereof, found in the bile of an animal (e.g., a human), including, by way of non-limiting example, cholic acid (Formula 2), cholate (Formula 3), deoxycholic acid (Formula 6), deoxycholate (Formula 7), hyodeoxycholic acid (Formula 12), hyodeoxycholate (Formula 13), glycoeholic acid (Formula 14), glycocholate (Formula 15), taurocholic acid (Formula 16), taurocholate (Formula 17) and the like. Taurocholic acid and/or taurocholate are referred to as TCA. Other exemplary low molecular weight bile acid biomarkers are known to those skilled in the art.

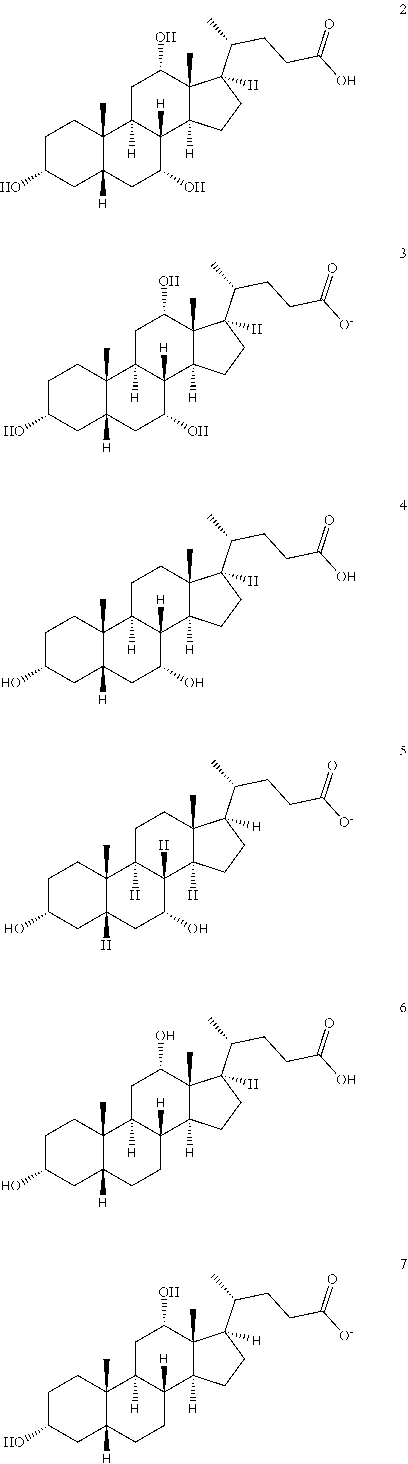

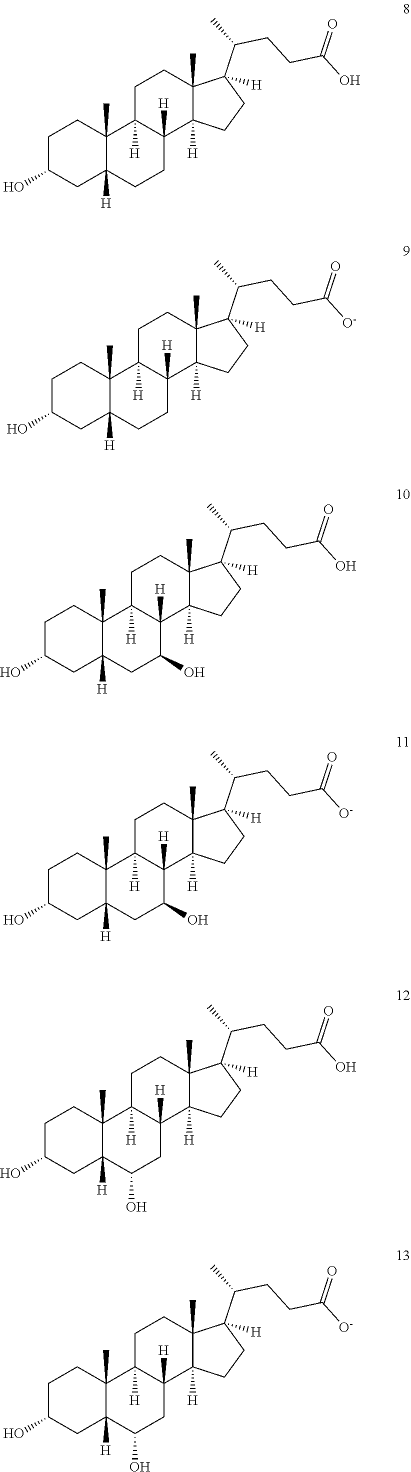

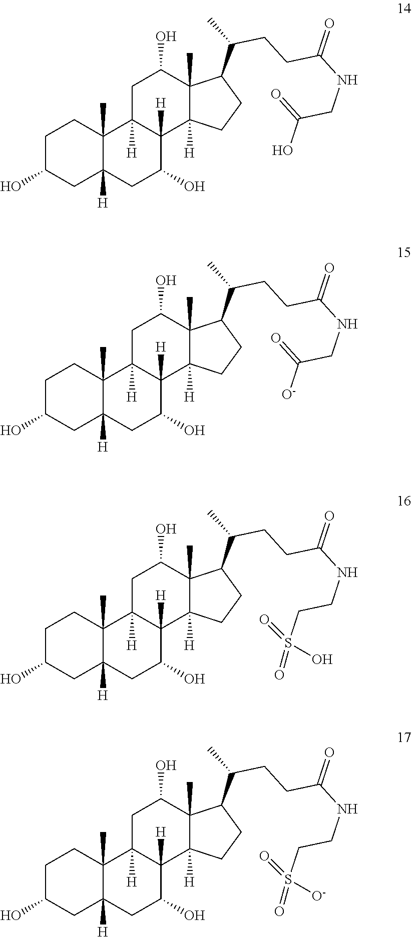

Any reference to a bile acid includes reference to a bile acid, one and only one bile acid, one or more bile acids, or to at least one bile acid. Therefore, the phrases “bile acid,” “bile salt,” “bile acid/salt,” “bile acids,” “bile salts,” and “bile acids/salts” are, unless otherwise indicated, utilized interchangeably. Much of the secreted bile acids are in the loon of conjugates with the amino acids taurine or glycine and/or conjugates with sulfate. The terms “conjugating,” “conjugation” and “conjugated” refer to the formation of a covalent bond. Conjugation of bile acids is catalyzed by enzymatic reactions that convert the bile acid to an acyl-CoA thioester then transfer the bile acid moiety from the acyl-CoA thioester to either glycine or taurine to form the respective bile acid conjugate. These additions substantially increase the acidity of the molecules and their solubility in water. At the physiological pH values in the intestines, the bile acid conjugates ionize and exist in salt form. In the conjugated state, the molecules cannot passively enter the epithelial cells of the biliary tract and small intestines. Therefore, any reference to a bile acid includes reference to a bile acid or a salt thereof. Furthermore, “bile acids” include bile acids conjugated to an amino acid (e.g., glycine or taurine). For example, the phrase “bile acid” includes cholic acid (Formula 2) conjugated with either glycine or taurine: (Formula 15) and taurocholate (Formula 17), respectively (and salts thereof).

Any reference to a bile acid includes reference to an identical compound naturally or synthetically prepared. Furthermore, it is to be understood that any singular reference to a component (bile acid or otherwise) includes reference to one and only one, one or more, or at least one of such components. Similarly, any plural reference to a component includes reference to one and only one, one or more, or at least one of such components, unless otherwise noted. Examples of low molecular weight bile acid metabolic biomarkers include, but are not limited to, those of Table 3:

TABLE 3

| Bile Acid | Type |
|---|---|
| Cholic acid | primary |
| Chenodeoxycholic acid | primary |
| α-Muricholic acid | primary |
| β-Muricholic acid | primary |
| ω-Murichoclic acid | primary |
| Deoxycholic acid | secondary |
| Lithocholic acid | secondary |
| Hyodeoxycholic acid | secondary |
| Ursodeoxycholic acid | secondary |
| Glycocholic acid | conjugated primary |
| Glycochenodeoxycholic acid | conjugated primary |
| Taurocholic acid | conjugated primary |
| Taurochenodeoxycholic acid | conjugated primary |
| Tauromuricholic acid | conjugated primary |
| Glycodeoxycholic acid | conjugated secondary |
| Glycolithocholic acid | conjugated secondary |
| Taurodeoxycholic acid | conjugated secondary |
| Taurolithocholic acid | conjugated secondary |
| Glycoursodeoxycholic acid | conjugated secondary |
| Tauroursodeoxycholic acid | conjugated secondary |

“Biological sample” refers to any sample that may contain relevant metabolites, including biological fluids such as, but not limited to, blood, urine, sweat, saliva, and sputum. The biological sample is obtained from the pediatric subject in a manner well-established in the art, “Blood” as used herein encompasses whole blood, blood plasma, and blood serum. The biological sample, like blood samples, may be analyzed without or after a pre-treatment. Examples of pre-treated blood samples are pre-treated blood, like. EDTA-blood, or EDTA-plasma, citrate-plasma, heparin plasma. The originally obtained (blood) samples or fractions thereof may be further modified by methods known in the art, as for example by fractionation or dilution. Fractionation tray be performed to remove constituents that might disturb the analysis. Dilution may be performed by mixing the original (blood) sample or fraction with a suitable sample liquid, like a suitable buffer, in order to adjust the concentration the constituents, as for example of the analyte. Such, modified (blood) samples exemplify samples “derived from” the original body fluid sample collected or isolated from the body of the individual.

“Chromatography” refers to a physical method of separation in which the components to be separated are distributed between two phases, one of which is stationary (stationary phase) while the other (the mobile phase) moves in a definite direction. The mobile phases can be aqueous or organic solvents, or mixtures thereof, as used in liquid chromatography or gasses such as helium, nitrogen or argon as used in gas chromatography. Chromatographic output data may be used for manipulation.

“Determining” refers to methods that include identifying the presence or absence of metabolic biomarkers in the sample, quantifying the amount of substance(s) in the sample, and/or qualifying the type of substance, “Determining” likewise refers to methods that include identifying the presence or absence of specific metabolic biomarkers. A “positive” reference concentration level of a metabolic biomarker means a level that is indicative of a particular disease state or phenotype. A “negative” reference concentration level of a metabolite means a level that is indicative of a lack of a particular disease state or phenotype. For example, a “heart failure-positive reference level” of a metabolic biomarker means a level of a metabolite that is indicative of a positive diagnosis of heart failure in a pediatric subject and a “heart failure negative reference level” of a metabolite means a level of a metabolite that is indicative of a negative diagnosis of heart, failure in a pediatric subject. A “reference concentration level” of a metabolite may be an absolute or relative amount or concentration of the metabolite, a presence or absence of the metabolite, a range of amount or concentration of the metabolite, a minimum and/or maximum amount or concentration of the metabolite, a mean amount or concentration of the metabolite, and/or a median amount or concentration of the metabolite; and, in addition, “reference levels” of combinations of metabolites may also be ratios of absolute or relative amounts or concentrations of two or more metabolites with respect, to each other or a composed value/score obtained by a statistical model.

“Heart failure” refers to a clinical syndrome resulting from structural or functional cardiac disorders that impair the ability of the heart to eject blood adequate for the needs of the body. The main manifestations in this syndrome are subjective symptoms of dyspnea (due to pulmonary congestion hence, congestive heart failure) and fatigue. These symptoms are associated with various measureable objective signs. The objective signs include: fluid retention (weight gain), decreased exercise tolerance (maximal walk distance), decrease in the total oxygen consumption, and changes in the hemodynamic indices (as decrease in the cardiac output increase in the left atrial pressure, increase in the end-diastolic pulmonary pressure). In small children and infants heart failure is associated with poor feeding, poor growth, enlarged liver, and listlessness. Heart failure is associated with changes in cardiac ejection fraction (systolic heart failure) and\or impairment in the cardiac filling (diastolic heart failure) that are measured by various imaging techniques. A prominent sign of heart failure is the development of lung congestion that is most commonly assessed by monitoring the thoracic impedance. One of skill in pediatric cardiology is familiar with pediatric heart failure scales such as the Ross Scale, American Heart Association Heart Failure Scales such as the New York American Heart Failure Scale, and the New York University Pediatric Heart Failure Index, all of which are incorporated herein by reference. In addition, one of ordinary skill would also be familiar with the American Heart. Association. The diagnosis of, and determination of stage or level of progression of, heart failure may also be based on one or more factors from those scales in combination with clinical judgement as guided by feeding, growth rate, and physical examination and treated by use of diuretics which is standard of care for pediatric subjects.

“Metabolite” refers to any compound produced or used during all the physical and chemical processes within the body that create and use energy or are involved in biosynthetic or catabolic processes which maintain the healthy homeostatic state of the organism. Such processes include digesting food and nutrients, eliminating waste through, breathing, circulating blood, and regulating temperature, mounting inflammatory and immune responses, etc. Metabolites also refer to compounds produced through the action of the gut microbiota. A metabolite may be an intermediate or product resulting from metabolism. Metabolites are often referred to as “small molecules.” Metabolites are molecules produced through metabolism in the body of a specified compound or salt thereof. Metabolites may be identified and quantified using analytical techniques known to those skilled in the art.

“Level” refers to a quantifiable amount of a metabolite in a sample. For example, the level may be a concentration level from a chromatography, nuclear magnetic resonance (NMR) spectroscopy, or mass spectrometry comprising analysis for a metabolite. The level of the metabolite in a sample may be expressed as arbitrary units related to concentration or as actual concentration units such as millimolar (mM), micromolar (μM) or picomolar (pM). The data may also be reported as ratios of metabolites or sets of metabolites. These data may be calculated from data from an assay and may be based on calibration data. A “different level” or “elevated level” of a metabolite refers to the amount or concentration of a metabolite in a sample from a subject compared to statistically validated thresholds, e.g., the amount of the metabolite in a sample(s) from individual(s,) that do not have heart failure, have heart failure (or a particular severity or stage of heart failure), have no symptoms of heart failure, or have other reference diseases other than a single ventricle heart. A “different level” or “elevated level” of a metabolite also may refer to the amount or concentration of a metabolite in a sample from a subject compared to statistically validated thresholds, e.g., the amount of the metabolite in a sample(s) from individual(s) that do not have heart failure, have heart failure (or a particular severity or stage of heart failure), have no symptoms of heart failure, or have other reference diseases. For example, a metabolite has an “elevated level” in the blood from a pediatric subject when the metabolite is present at a higher concentration in the subject's blood sample than in blood from a subject who does not have heart failure. “Change” in the level of one or more metabolic biomarkers refers to an increase or a decrease of by about 1.2-fold or greater in the level of the metabolic biomarkers as determined in a biological sample obtained from the subject as compared to the reference level of the one or more metabolic biomarkers. In one embodiment, the change in level is an increase or decrease by about 1.2- fold. Fold change is calculated as (New value)/(Old value),

“Low molecular weight metabolic biomarker” refers to endogenous organic compounds of a cell, an organism, a tissue or being present in body liquids, in particular blood, and in extracts or fractions obtained from blood such as plasma. Typical examples of metabolites are compounds from chemical classes including carbohydrates, amino acids, organic acids acylcarnitines, bile acids, lipids, phospholipids, sphingolipids and sphingophospholipids, cholesterols, steroid hormones and other compounds known to those skilled in the art. This includes any substance produced by metabolism or by a metabolic process and any substance involved in metabolism. In particular, suitable metabolites are described in Table 4. More particular, they may have a molecular weight typically of up to 1500 Dalton, as for example in the range of 50 to 1500 Dalton. As noted above, a “metabolite” is an intermediate or product resulting from metabolism. Metabolites are often referred to as “small molecules.” Metabolites are molecules produced through metabolism in the body of a specified compound or salt thereof. Metabolites may be identified and quantified using analytical techniques known to those skilled in the art. “Biomarker” refers to concentration data of at least one, as for example 1, 2, 3, 4, 5, 6, 7, 8, 9 or 10, 11, 12, 13, 14, 15, 16, 17, 18, 19, 20, 21. 22, 23, 24, 25 metabolites (also designated as a “panel” of metabolites, “signature” of metabolites, “model” or “profile” using quantitative data or concentration data directly or processed by any mathematical transformation (e.g. by log transformation, unit variance scaling, Pareto scaling or a classification method) and evaluated as an indicator of biologic processes or responses to a therapeutic intervention associated with heart failure in pediatric subjects. “Biomarker” is intended to also comprise ratios between two or more metabolites/biomarkers. Thus, the tent) “biomarker” may also encompass the ratio of the amount of two or more metabolites. Exemplary low molecular weight metabolic biomarkers include, but are not limited to metabolites in the following categories or classes: acylcarnitines, amino acids, bile acids, lipids, steroids, products of benzoate metabolism, product of aromatic amino acid synthesis and tricarboxylic acid cycle (also called the Krebs cycle) intermediates. A low molecular weight metabolic biomarker is not a protein.

TABLE 4

| Metabolite | Chemical Class | Metabolic Pathway |
|---|---|---|
| Methylmalonylcarnitine | acylcarnitine | fatty acid oxidation |
| Hydroxyvalerylcarnitine | acylcarnitine | fatty acid oxidation |
| Pimeloylcarnitine | acylcarnitine | fatty acid oxidation |
| Dodecanedioylcarnitine | acylcarnitine | fatty acid oxidation |
| Hydroxyhexadecanoylcarnitine | acylcarnitine | fatty acid oxidation |
| Aspartic acid | amino acid | amino acid metabolism |
| Glutamic acid | amino acid | amino acid metabolism |
| Histidine | amino acid | amino acid metabolism |
| 3-Indolacetic acid | Indoles | amino acid, gut microbial metabolism |
| Threonine | amino acid | amino acid metabolism |
| α-Aminobutyric acid | amino acid | amino acid metabolism |
| γ-aminobutyric acid | amino acid | amino acid metabolism |
| Nitro-Tyrosine | amino acid | amino acid metabolism |
| Ornithine | amino acid | amino acid and nitrogen metabolism |
| Betaine | amino acid | amino acid and one-carbon metabolism |
| Glycocholic acid | bile acid | primary bile acid metabolism |
| Glycochenodeoxycholic acid | bile acid | primary bile acid metabolism |
| Glycoursodeoxycholic acid | bile acid | secondary bile acid metabolism |
| Taurocholic acid | bile acid | primary bile acid metabolism |
| Taurochenodeoxycholic acid | bile acid | primary bile acid metabolism |
| dehydroepiandrosterone sulfate | hormone | cholesterol, hormone metabolism |
| Hippurate | organic acids | dietary metabolism |
| Succinate | organic acids | tricarboxylic acid cycle |
| Citrate | organic acids | tricarboxylic acid cycle |
| FA(20.2) | fatty acid | fatty acid metabolism |
| PC(32.0) | phosphocholine | lipid metabolism |
| DG(16.0:16.0) | diglyceride | lipid metabolism |
| DG(18.1:18.1) | diglyceride | lipid metabolism |
| DG(18.1:18.2) | diglyceride | lipid metabolism |
| TG(16.0:36.3) | triglyceride | lipid metabolism |
| TG(16.0:36.6) | triglyceride | lipid metabolism |
| TG(18.0:36.2) | triglyceride | lipid metabolism |
| TG(18.0:36.3) | triglyceride | lipid metabolism |
| TG(18.1:36.1) | triglyceride | lipid metabolism |
| TG(18.1:36.2) | triglyceride | lipid metabolism |
| TG(18.1:36.3) | triglyceride | lipid metabolism |
| TG(18.1:36.4) | triglyceride | lipid metabolism |
| TG(18.1:36.5) | triglyceride | lipid metabolism |
| TG(18.2:36.1) | triglyceride | lipid metabolism |
| TG(18.2:36.2) | triglyceride | lipid metabolism |
| TG(18.2:38.4) | triglyceride | lipid metabolism |
| TG(18.2:38.5) | triglyceride | lipid metabolism |
| TG(20.5:34.1) | triglyceride | lipid metabolism |
| TG(20.5:34.2) | triglyceride | lipid metabolism |
| TG(20.5:36.3) | triglyceride | lipid metabolism |

Note: parenthetical values indicate the length, of fatty acid side chains. For FA the first number is the length of the side chain and the second number separated by a period is the degree of unsaturation in that side chain. Degree of unsaturation is the same as the number of double bonds. For PC, the number includes the total number of carbons in two fatty acid side chains. For DG the numbers indicate the chain lengths and degrees of unsaturation of the two fatty acid side chains. For TGs the first number indicates the chain length and degree of unsaturation of one chain and the second numbers indicate the sum of the chain lengths and degrees of unsaturation of the second and third fatty acid side chains.

Mass Spectrometry (MS) is a technique for measuring and analyzing molecules that involves ionizing and/or fragmenting a target molecule, then analyzing the fragments, based on their mass/charge ratios, to produce a mass spectrum that serves as a “molecular fingerprint.” Several commonly used methods to determine the mass to charge ratio of an ion are known, some measuring the interaction of the ion trajectory with electromagnetic waves, others measuring the time an ion takes to travel a given distance, or a combination of both. The data from these fragment mass measurements can be searched against databases to obtain definitive identifications of target molecules.

Nuclear magnetic resonance (NMR) spectroscopy is a technique for determining the molecular structure and concentration of molecules. In the NMR analysis of metabolites, the biological samples are maintained in solution and then placed in a large magnetic field. The magnetic field is typically generated by a cryogenically cooled superconducting magnet, but lower magnetic fields including those generated by room temperature electromagnets or rare earth magnets can also be used. The samples are exposed to a band of radiofrequency (RF) waves which are absorbed by the molecules and excite the nuclei of the molecules. The typical focus of these experiments is on the excitation of hydrogen nuclei (also referred to as protons) and nuclei of carbon, in particular the C-13 isotope. After the RF excitation is turned off, a receiver coil then detects the RF energy that is released by the nuclei as they return from the excited state. Following Fourier transformation of these signals, an NMR spectrum is generated containing spectral peaks that, in different combinations, represent the sum of the metabolites in the sample. The resulting NMR spectrum can then be compared with reference spectra to both identify and quantify the different metabolites in the sample.

“Single ventricle” refers to those heart physiologies unable to create a circulation in series due to anatomical defects. That is, single ventricle heart includes a cardiac defect in which there is only one functioning ventricle; where the single ventricle may be a morphological right or left ventricle, or indeterminate; such as due to an underdeveloped chamber, valve, or outflow tract, or there may be two good-sized ventricles where the inflow and/or outflow tracts cannot be separated. Specific examples may include hypoplastic ventricles (such as hypoplastic left heart syndrome, or hypoplastic right heart); AV valve atresia (tricuspid atresia); abnormal inlet (double inlet left ventricle) and inability to septate (heterotaxy syndrome with left atrial isomerism, unbalanced atrioventricular canal and double outlet right ventricle).

“Pediatric” refers to a population of subjects ranging between a newborn and less than 18 or about 18 years of age. A pediatric subject can include a subject that begins a course of treatment according to the disclosed methods before turning about 18 years of age, even if the subject continues treatment beyond 18 years of age. More specifically, within the population of “pediatric” subjects, neonates may be defined as newborn to 1 month in age (including newborn to 2 weeks, newborn to three weeks), infants may be 1 month to less than 2 years of age (including ages in, months such as 2, 3, 4, 5, 6, 7, 8, 9, 10, 11, 12, 13, 14, 15, I6, 17, 18, 19, 20, 21, 22, and 23), toddlers may be 2 years to less than 6 years of age (including 3 years, 4 years, and 5 years), and school age may refer to subjects 6-18 years of age (including 7 years, 8 years, 9 years, 10 years, 11 years, 12 years, 13 years, 14 years, 15 years, 16 years and I 7 years). Pediatric does not mean adult.

“Ratio” refers to a calculable relationship used to compare amounts of the concentration levels of two or more validated low molecular weight metabolic biomarkers. A ratio may be a direct proportion or inverse proportion (e.g., a first amount divided by a second amount or the second amount divided by the first amount, respectively). A ratio may be weighted and/or normalized (either the numerator, the denominator, or both). The two amounts may be physical quantities or arbitrary values that correspond to physical quantities. For example, a ratio may be calculated from two concentration levels (i.e., in arbitrary units) in two biomarkers (e.g., validated low molecular weight metabolic biomarkers) measured by a mass spectrometry technique.

“Score” denotes a value, in particular a quantitative value, generated from metabolic biomarker data by means of any mathematical transformation or by subjecting to any mathematical equation and comparing these data to data or mathematically transformed or processed data of a reference or control population.

“Subject” refers to a human subject as well as a non human subject such as a non human mammal. Thus, various veterinary applications are contemplated in which case the subject may be a non-human mammal (e.g., a feline, a porcine, an equine, a bovine, and the like). The concepts described are also applicable to plants. The term does not denote a particular age or sex and, thus, includes pediatric and newborn subjects, whether male or female. “Normal control subjects” or “normal controls” means healthy subjects who are clinically free of single ventricle heart and heart failure. “Normal control sample” or “control sample” refers to a biological sample that has been obtained from a normal control subject.

“Validated” refers to a statistically significant difference in a level of a metabolite biomarker in a biological sample from a subject and a reference or control level of at least about 5%, or greater, e.g., at least about 10%, 15%, 20%, 25%. When performing multivariate statistical analysis during, biomarker discovery in metabolomics, corrected p-values are often used to correct for multiple hypothesis testing in order to reduce false discoveries, such as the use of a false discovery rate (q<0.05) or a more conservative Bonferroni correction. The determination of statistical significance is well-established in the art. Statistical significance is attained when a p value is less than the significance level. The p-value is the probability of observing an effect given that the null hypothesis is true whereas the significance or alpha level is the probability of rejecting the null hypothesis given that it is true.

In one embodiment, the concentration level of the metabolic biomarker in the sample obtained from the subject is increased as compared to the reference level of the biomarker. Suitable biomarkers that indicate a risk that a single ventricle pediatric subject will progress to heart failure when the concentration level increases can be, for example, one or more biomarkers as listed in Table 4, and combinations thereof.

In another embodiment, the concentration level of the metabolic biomarker in the sample obtained from the subject is decreased as compared to the reference level of the metabolic biomarker. Suitable biomarkers that indicate a risk that a single ventricle pediatric subject will progress to heart failure when the concentration level decreases as compared to the expression level have been found to include, for example, one or more biomarkers as listed in Table 4, and combinations thereof.

Once a biological sample is obtained, it is analyzed to determine the concentration level of the selected validated metabolite biomarker(s) in the sample. Before analysis, the sample may be subject to processing such as extraction, filtration, centrifugation or other sample preparation techniques to provide a sample that is suitable for further analysis For example, biological fluids may be filtered or centrifuged (e.g., ultracentrifugation) to remove solids from the sample to facilitate analysis. As one of skill in the art will appreciate, biomarker level may be determined using, one of several techniques established in the art that would be suitable for detecting such biomarkers, e.g. polar metabolites, in a biological sample, including mass spectrometry, chromatographic techniques such as high performance liquid chromatography and gas chromatography, immunoassay or enzyme-based assays with colorimetric, fluorescence, or radiometric detection. As one of skill in the art will appreciate, metabolite biomarkers may be analyzed directly or may be chemically derivatized for analysis, and may be analyzed by comparison against stable-isotope internal standards.

In some embodiments, metabolite biomarker detection using a mass spectrometry (MS)-based method is used. Suitable MS-based methods for use include direct infusion-mass spectrometry, electrospray ionization (ESI)-MS, desorption electrospray ionization (DESI)-MS, direct analysis in real-time (DART)-MS, atmospheric pressure chemical ionization (APCI)-MS, electron impact (EI) or chemical ionization (CI), as well as MS-based methods coupled with a separation technique, such as liquid chromatography (LC-MS), gas chromatography (GC-MS), or capillary electrophoresis (CE-MS) mass spectrometry,

In some embodiments, a computer-based analysis program is used to translate the raw data generated by the detection assay (e.g., the presence, absence, or, amount of a heart failure specific metabolite) into data of predictive value for a clinician. The clinician can access the predictive data using any suitable means. Thus, in some embodiments, the clinician, who is not likely to be trained in metabolites analysis, need not understand the raw data. The data is presented directly to the clinician in its most useful form. The clinician is then able to immediately utilize the information in order to optimize the care of the subject. Any method capable of receiving, processing, and transmitting the information to and from laboratories conducting, the assays, information providers, medical personal, and subjects is contemplated. For example, in some embodiments, a sample (e.g., a biopsy or a blood, urine or plasma sample) is obtained from a subject and submitted to a profiling service (e.g., clinical lab at a medical facility, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling, center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (e.g., metabolic profile), specific for the diagnostic or prognostic information desired for the subject.

The profile data is then prepared in a format suitable for interpretation by a treating clinician. For example, rather than providing raw data, the prepared format may represent a diagnosis or risk assessment (e.g., likelihood of heart failure being present, aggressiveness of the heart failure, risk of developing heart failure in the future) for the subject, along with recommendations for particular treatment options. The profile data, may also be used by the treating clinician to measure response to a therapy intended to treat, for example, heart failure, elevated venous pressures, elevated pulmonary artery pressures, or systemic arterial hypertension. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or subject. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers. In some embodiments, the subject is able to directly access the data using the electronic communication system. The subject may choose further intervention or counseling based on the results. In some embodiments, the data is used for research use. For example, the data may be used to further optimize the inclusion or elimination of markers as useful indicators of a particular condition or stage of disease.

In some embodiments, diagnostic kits for assessing an attribute of heart failure in a pediatric subject having a single ventricle heart, where the attribute of heart failure is associated with a low molecular weight metabolic biomarker profile, includes; a container with at least, one validated low molecular weight metabolic biomarker internal standard having a purity greater than 98.0%, a biological sample receiving vessel, and a sealing member configured to seal the sample receiving vessel after receiving the biological sample.

In embodiments, the low molecular weight metabolic biomarker internal standard may be one or more of the low molecular weight metabolic biomarkers described above. In embodiments, the low molecular weight metabolic biomarker may be one or more of those listed in Table 4.

In embodiments, the biological sample receiving vessel may be an ampule, a bottle, a vial, a squeeze bottle, a test tube, a micro-tube, a cuvette, a petri dish, or the like. The biological sample receiving vessel may be made of glass, plastics (such gas polyethylene terephthalate), aluminum, steel, composites, or a combination of two or more thereof.

In embodiments, the sealing member may be a press-on cap, a screw-on cap, a rubber stopper, a cork stopper, a rubber septum, a portion of sealing tape, a portion of parafilm, and a combination of two or more thereof.

Aspects of the present disclosure can be described as embodiments in any of the following enumerated clauses. It will be understood that any of the described embodiments can be used in connection with any other described embodiments to the extent that the embodiments do not contradict one another.

In an aspect, either alone or combined with any other aspect, a method to assess a pediatric subject having a single ventricle heart includes: determining a concentration level of one or, more validated low molecular weight metabolic biomarkers in a biological sample from the subject; identifying a difference between the determined concentration level of the one or more metabolic biomarkers and a reference Concentration level of the one or more metabolic biomarkers; and, assessing the pediatric subject based on the identified difference.

In an aspect, either alone or combined with any other aspect, the reference concentration level is determined from a healthy subject, a subject having single ventricle heart failure, or a combination thereof.

In an aspect, either alone or combined with an other aspect, assessing the pediatric subject includes or is determining a stage in progression to heart failure.

In an aspect, either alone or combined with any other aspect the stage is stable heart function.

In an aspect, either alone or combined with any other aspect, the stage is congenital heart failure.

In an aspect, either alone or combined with any other aspect, assessing the pediatric subject includes or is predicting heart failure.

In an aspect, either alone or combined with any other aspect, the pediatric subject has been diagnosed as having heart failure and assessing the pediatric subject includes or is determining a stage in progression of the heart failure.

In an aspect, either alone or combined with any other aspect, assessing the pediatric subject includes or is determining a risk of developing heart failure.

In an aspect, either alone or combined with any other aspect, assessing the pediatric subject includes or is differentiating single ventricle pediatric subjects not having heart failure from single ventricle pediatric subjects having heart failure.

In an aspect, either alone or combined with any other aspect, the method further includes calculating a ratio of the concentration levels of one or more validated low molecular weight metabolic biomarkers.

In an aspect, either alone or combined with any other aspect, the method further includes determining at least one of a presence of, a risk of, and a stage of heart failure based, at least in part, on a ratio of the concentration levels of one or more validated low molecular weight metabolic biomarkers.

In an aspect, either alone or combined with any other aspect, the pediatric subject is a pediatric human subject.

In an aspect, either alone or combined with any other aspect, the pediatric subject is an infant human subject.

In an aspect, either alone or combined with any other aspect, the pediatric subject is a male.

In an aspect, either alone or combined with any other aspect, the pediatric subject is a female.

In an aspect, either alone or combined with any other aspect, the biological sample comprises a blood plasma sample.

In an aspect, either alone or combined with any other aspect, the one or more validated low molecular weight metabolic biomarkers comprises a panel of from five to ten validated low molecular weight metabolic biomarkers.

In an aspect, either alone or combined with any other aspect, the low molecular weight metabolic biomarker is selected from one or more of the low molecular weight metabolic biomarkers in Table A.

In an aspect, either alone or combined with any other aspect, the pediatric subject is undergoing a treatment for single ventricle heart.

In an aspect, either alone or combined with any other aspect, the pediatric subject is undergoing a treatment for single ventricle heart and the treatment is continued, discontinued, or altered based on presencem absence, or the concentration level of the one or more low molecular weight metabolic biomarkers.

In an aspect, either alone or combined with any other aspect, the method further includes providing a recommended treatment based on presence, absence, or concentration level of the one or more low molecular weight metabolic biomarkers; and administering the treatment to the pediatric subject.

In an aspect, either alone or combined with any other aspect, the low molecular weight metabolic biomarker comprises an acylcarnitine.

In an aspect, either alone or combined with any other aspect, the low molecular weight metabolic biomarker comprises a dicarboxylated acylcarnitine or a hydroxylated acylcarnitine.

In an aspect, either alone or combined with any other aspect, the low molecular weight metabolic biomarker comprises an acylcarnitine selected from the group consisting of methylmalonylcarnitine, pimeloylcarnitine, dodecanedioylcarnitine and hydroxyhexadecanoylcarnitine.

In an aspect, either alone or combined with any other aspect, the low molecular weight metabolic biomarker comprises an amino acid.

In an aspect, either alone or combined with any other aspect, the low molecular weight metabolic biomarker comprises a canonical amino acid or a non-canonical amino acid.

In an aspect, either alone or combined with any other aspect, the low molecular weight metabolic biomarker comprises an amino acid selected from the group consisting of aspartate, glutamate, histidine, threonine, nitro-tyrosine, ornithine, α-aminobutyric acid and γ-aminobutyric acid.

In an aspect, either alone or combined with any other aspect, the low molecular weight metabolic biomarker comprises a bile acid.

In an aspect, either alone or combined with any other aspect, the low molecular weight metabolic biomarker comprises a secondary bile acid or a conjugated bile acid.

In an aspect, either alone or combined with any other aspect, the low molecular weight metabolic biomarker comprises a bile acid selected from the group consisting of glycoeholic acid, glycochenodeoxycholic acid, glycoursodcoxycholic acid, taurocholic acid and taurochenodeoxycholic acid.

In an aspect, either alone or combined with any other aspect, the low molecular weight metabolic biomarker comprises a lipid.

In an aspect, either alone or combined with any other aspect, the low molecular weight metabolic biomarker comprises a triacylglycerol or a triglyceride.

In an aspect, either alone or combined with any other aspect, the low molecular weight metabolic biomarker comprises betaine.

In an aspect, either alone or combined with any other aspect, the low molecular weight metabolic biomarker comprises a steroid.

In an aspect, either alone or combined with any other aspect, the low molecular weight Metabolic biomarker comprises an androstane steroid.

in an aspect, either alone or combined with any other aspect, the low molecular weight metabolic biomarker comprises the androstane steroid dehydroepiandrosterone sulfate.

In an aspect, either alone or combined with any other aspect, the low molecular weight metabolic biomarker comprises a product of benzoate metabolism.

In an aspect, either alone or combined with any other aspect, the low molecular weight metabolic biomarker comprises hippurate.

In an aspect, either alone or combined with any other aspect, the low molecular weight metabolic biomarker comprises a product of aromatic amino acid synthesis.

In an aspect, either alone or combined with any other aspect, the low molecular weight metabolic biomarker comprises 3-indoleacetate.

In an aspect, either alone or combined with, any other aspect, the low molecular weight metabolic biomarker comprises a Krebs cycle intermediate.

In an aspect, either alone or combined with any other aspect, the low molecular weight metabolic biomarker comprises citrate and succinate.

In an aspect, either alone or combined with any other aspect, the concentration level of the one or more low molecular weight metabolic biomarkers in the biological sample is determined by mass spectrometry.

In an aspect, either alone or combined with any other aspect, the concentration level of the one or more low molecular weight metabolic biomarkers in the biological sample is determined by a chromatography and/or spectrometry method.

In an aspect, either alone or combined with any other aspect, the concentration level of the one or more low molecular weight metabolic biomarkers is determined by chromatography comprising GC, LC, HPLC, and UPLC spectroscopy comprising UV/Vis, IR, and NMR; and mass spectrometry comprising ESI-QqQ, ESI-QqTOF, MALDI-QcQ, MALDI-QqTOF, and MALDI-TOF-TOF.

In an aspect, either alone or combined with any other aspect, the subject has not been diagnosed with heart failure.

In an aspect, either alone or combined with any other aspect, the method further includes altering a treatment course of action.

In an aspect, either alone or combined with any other aspect, the method further includes determining a treatment course of action.

In an aspect, either alone or combined with any other aspect, a diagnostic kit for assessing an attribute of heart failure in a pediatric subject having a single ventricle heart disease, where the attribute of heart failure is associated with a low molecular weight metabolic biomarker profile, includes: a container comprising at least one validated low molecular weight metabolic biomarker internal standard having a purity greater than 98.0%, where the container is configured to receive a biological sample from the pediatric subject and to be sealed with a sealing member after receiving the biological sample.

In an aspect, either alone or combined with any other aspect, a diagnostic kit for assessing an attribute of heart failure in a pediatric subject having a single ventricle heart disease, where the attribute of heart failure is associated with a low molecular weight metabolic biomarker profile, includes: a container comprising at least one validated low molecular weight metabolic biomarker internal standard having a purity greater than 98.0%, a sample receiving vessel for receiving a biological sample, and a sealing member for sealing the sample receiving vessel after receiving the biological sample.

In an aspect, either alone or combined with any other aspect, a method to identify a candidate low molecular weight metabolic biomarker that differentiates a pediatric subject having a single ventricle heart without heart failure and a pediatric subject that has single ventricle heart with heart failure, includes: obtaining a first biological sample from a pediatric subject having a single ventricle heart without heart failure and a second biological sample from a pediatric subject having a single ventricle heart with heart failure; determining a concentration level of one or more low molecular weight metabolic biomarkers in the first biological sample and the second biological sample; and, identifying one or more validated low molecular weight metabolic biomarkers that are present in a statistically significant, different level in the first and second biological samples, where the different level is characterized by an area under the receiver operator characteristic (ROC) curve (AUC) ranging from 0.60 to 1.00.

### EXAMPLES

Study Cohort: This study was approved by the Indiana University School of Medicine Institutional Review Board. From December 2018 to November 2019. pediatric subjects with SV were recruited from the pediatric cardiology clinics and inpatient cardiology service at Riley Hospital for Children, Pediatric subjects without heart disease served as controls (Con) and were recruited from the pediatric clinics. Management of subjects with SV was based on the standard of care (SOC), and the diagnosis of heart failure was based on clinical judgement of the attending cardiologist for that subject. Inclusion criteria far Con included structurally normal hearts. Exclusion criteria for both cohorts were the absence of metabolic or systemic diseases, such as diabetes, and the subject was not pregnant. All participants gave written informed consent for their participation in the study.

NMR-based metabolomics: Samples for NMR analyses were prepared using established protocols. Spectra were acquired on a Balker AVANCE III, 700 MHz NMR equipped with a cryogenically-cooled probe. The untargeted NMR analyses quantified 26 metabolites. The data was processed and analyzed using the Chenomx NMR Processor and Profilers software packages (Chenomx inc., Edmonton, Alberta, Canada).

Targeted MS-based Inetabolomics: The targeted MS experiments used the Biocrates Q500 kit run on an AB Sciex 5500 QTRAP with an Agi lent 1290 UPLC (Biocrates AG. Innsbruck, Austria). Preparation of serum samples followed vendor protocols. This assay yielded quantitative measures of 495 metabolites. Data processing was carried out using the Biocrates MetIDQ software.

Data integration and biomarker analysis: Integration, statistical analysis and visualization of the metabolomics data was carried out using the VIIME platform along with scripts written in the R programming language. Log transformation and Pareto scaling was applied to both the NMR and MS datasets. The receiver operator characteristic (ROC) analysis was carried out in MetaboAnalyst (version 5.0, ref) using the multivariate exploratory analysis functions. Briefly, ROC curves were generated by Monte-(Tarlo cross-validation with classification and ranking carried out by Partial Least Squares Discriminant Analysis (PLSDA) using two latent variables.

Twenty-six pediatric subjects provided written consent and were enrolled in the study. Pediatric subjects were not fasted for this study. Ten pediatric subjects had no heart disease and were enrolled in the Con group. Sixteen pediatric subjects bad a diagnosis of SV of which 8 had a diagnosis of hypoplastic left heart syndrome (“HLHS”), and 8 had a diagnosis of other forms of SV including double inlet left ventricle, tricuspid atresia, unbalanced atrioventricular canal, and double outlet right ventricle. Of those 16 pediatric subjects with SV, 9 were diagnosed with congestive heart failure (“HP”) based on physical examination and required diuretic support. This cohort was designated as SVHF. Seven single ventricle patients did not have a diagnosis of HF, were not on diuretic support, and were designated as SV. The baseline characteristics for each of the three groups are shown in Table 5. No significant differences in age, sex, height, or weight existed between all 3 groups. In addition, there were no significant differences in the anatomic diagnosis, age at surgery, or type of surgical palliation between the SV and SVHF cohorts. The mean oxygen saturation was not different between SV groups (92.9% vs 92.1%). Cardiac troponin I (cTnI) and C-reactive protein (CRP) were measured in 5 of 7 SV subjects, and 8 of 9 SV′FI subjects, The CRP was normal in all subjects except for one child (2.2 years) at 2.9 mg/dL, and the cTnI was <0.03 ng/mL in all subjects measured. Medication use between the cohorts was not different except for diuretic use in the SVHF group (p<0.0001). Within the SVHF cohort, 3 had protein losing enteropathy (PLE), and 3 required heart transplantation at a later date, whereas no SV pediatric subject had PLE, or heart transplantation.

TABLE 5

| | | | |
|---|---|---|---|
| Control (n = 10) | SV (n = 7) | SVHF (n = 9) | P-Value |

Epidemiological Background

| | | | | | | | |
|---|---|---|---|---|---|---|---|
| Age (years) | 9.16 | (2.2-14.3) | 11.01 | (7.5-15.3) | 8.82 | (2.8-19.7) | 0.64 |
| Sex (% male) | 3 | (30%) | 5 | (71%) | 3 | (33%) | 0.20 |
| Height (cm) | 137.1 | (90.5-171) | 141.6 | (122-176.3) | 119.8 | (93.7-158.5) | 0.18 |
| Weight (kg) | 40.15 | (16.9-72.6) | 41.07 | (24.7-87.3) | 29.8 | (13.3-88.5) | 0.52 |
| BMI (kg/m²) | 19.75 | (14.2-29.1) | 19.41 | (15.2-28.1) | 18.69 | (14.1-35.2) | 0.91 |

| | | | | | | |
|---|---|---|---|---|---|---|
| Diagnosis | Normal heart | | | | | |
| HLHS | | 3 | (43%) | 5 | (56%) | 0.64 |
| DILV/TA/AVC/DORV | | 4 | (57%) | 4 | (44%) | 1.0 |

Surgical Procedure

| | | | | | |
|---|---|---|---|---|---|
| Fontan Extra-Cardiac | 0 | (0%) | 3 | (33%) | 0.10 |
| Fontan Lateral Tunnel | 7 | (100%) | 6 | (67%) | 0.10 |
| Age at Fontan (months) | 28.3 | (11-57) | 36.44 | (18-44) | 0.29 |

Medications

| | | | | | |
|---|---|---|---|---|---|
| β-Blocker | 1 | (14%) | 3 | (33%) | 0.42 |
| ACE-inhibitor/ARB | 4 | (57%) | 7 | (78%) | 0.41 |
| Digoxin | 1 | (14%) | 4 | (44%) | 0.22 |
| Diuretic | 0 | (0%) | 9 | (100%) | <0.0001 |
| Aspirin | 5 | (71%) | 7 | (78%) | 0.79 |

Table 5 shows multi-group comparisons by one-way Anova, and two-group comparisons by unpaired t-test with the exception of Diuretic use (Mann-Whitney test). Continuous measurements are reported as mean and range (parenthesis), whereas categorical measurements are reported as total and percentage (parenthesis). Unadjusted P-values are presented in the right hand column with significance set at the 0.05 level. AVC=unbalanced atrioventricular canal, DILV=double inlet left ventricle, DORV=double outlet right ventricle, HLHS=hypoplastic left heart syndrome, and TA=tricuspid atresia.

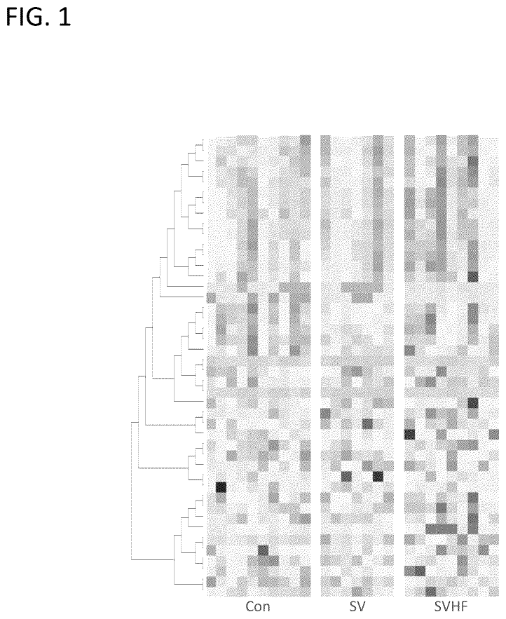

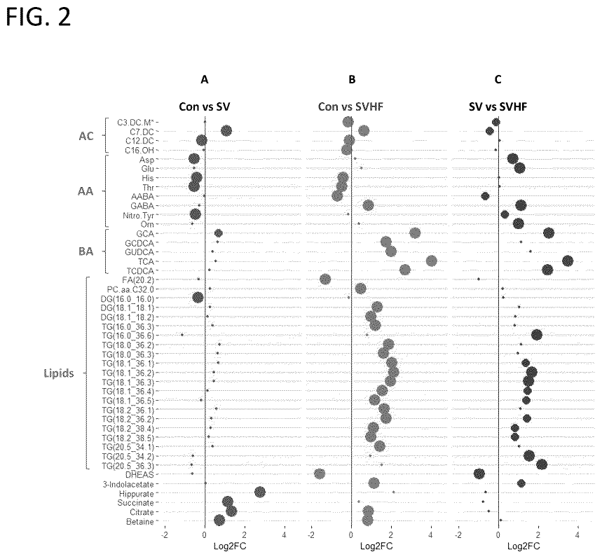

Metabolomics analysis yields distinct metabolic phenotypes of SV and SVHF. Integration of the NMR and MS data revealed a set of 44 metabolites for which there was a significant difference in at least one of the inter-group comparisons. A heatmap of these metabolites is shown in FIG. 1. FIG. 1 was generated using a hierarchical analysis of the metabolites and clearly shows a difference in the metabolite patterns for the three groups. To further detail the metabolic differences between the three groups, the changes of the individual metabolites are represented as forest plots in FIG. 2. A comparison of Con versus SV pediatric subjects is shown in column A of FIG. 2 with the log 2-fold change for each metabolite shown. Columns B and C of FIG. 2 present similar comparisons of Con versus SVIIF, and SV versus SV F, respectively.

A set of 4 acylcarnitines (ACs) were found to be significantly altered, including dicarboxylated (DC) and hydroxylated (OH) species. The short chain methylmalonylearnitine (C3-DC-M) was slightly reduced in SVHF compared to both controls and SV. The medium chain pimeloylcarnitine (CLDC) was increased in both SV and SVHF compared with controls. Trends toward reduced levels of these short and medium chain ACs were observed in SVHF versus SV. The longer chain dodecanedioylcarnitine (C12-DC) was reduced in both SV and SVHF, and the hydroxyhexadecanoyicarnitine (C16-OH) was significantly reduced only in the SVHF group. For these longer chain ACs no significant difference was observed between SV and SVHF cohorts. Note that C3-DC-M is isobaric with the other short chain AC, hydroxyvalerylcarnitine (C5.0H) and is an alternative assignment in FIG. 2.

A set of 8 amino acid-related compounds were significantly altered. Of these, four were canonical amino acids; aspartate (Asp), glutamate (Glu), histidine (His) and threonine (Thr). The significant changes included reductions in both SV and SVHF compared with controls. The SVHF pediatric subjects were characterized by increased levels of Asp and Glu compared with SV. The four non-standard amino acids include nitro-tyrosine, ornithine, α-amnobutyric acid (ARBA) and γ-aminobutyric acid (GABA). AABA was significantly reduced in SVHF and trended downward in comparing SVHF with SV. In contrast, GABA was significantly increased in SVHF compared to both Con and SV. A significant reduclion in nitro-tyrosine is only observed in the Con versus SV comparison along with a trending reduction in SVHF compared with SV. Ornithine demonstrated a significant increase in SVHF compared with Con.

A set of five bile acids were found to be altered including the secondary bile acid glycocholic acid (GCA) and the conjugated pritnaty bile acids glycochenodeoxycholic acid (GCDCA), glycoursodeoxycholic acid (GUDCA), taurocholic acid (TCA) and taurochenodeoxycholic acid (TCDCA). All alterations involve increases With a trending increase in GCA in the SV group while four of the five are significantly elevated in the SVHF group. Distinguishing the SVHF horn SV groups are significant increases in three bile acids.

Alterations in lipids are represeined by changes in 21 metabolites, with 16 of these being triacylglycerols (IGs). Interestingly, there were no changes in the TGs in the SV group, but 13 of these were significantly increase in the SVHF group. A set of 5 significant increases in TGs were observed in the SVHF group compared with SV, along with trending increases in 6 others.

Additional metabolic changes were found which suggest potential effects on hormone signaling, energy metabolism and one-carbon metabolism. A significant decrease in the androstane steroid, dehydroepiandrosterone sulfate (DHEAS) was observed between Con versus SVHF which persisted when comparing SV versus SVHF. Additional changes were observed in 3-indoleacetate and hippurate. The Krebs cycle intermediates citrate and succinate were both increased in Con versus SV, whereas only the increase in citrate was observed in the Con versus SVHF. Neither Krebs cycle intermediate was different between SV or SVHF. Betaine functions mainly as a methyl donor that plays a significant role in a number of processes including liver function. Significant increases in betaine were observed in the Con versus SV, and Con versus SVHF comparisons.

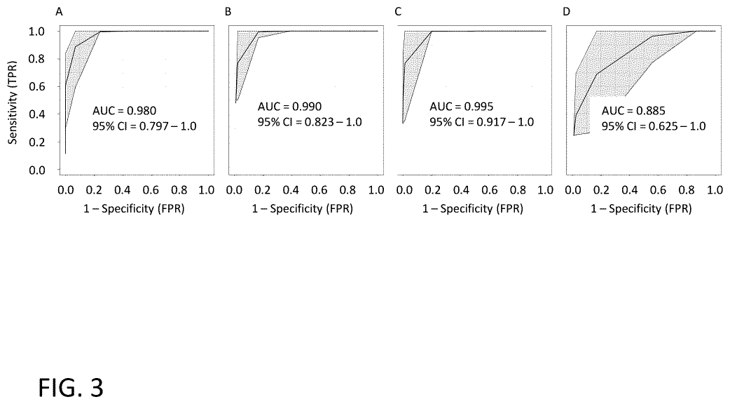

Diagnostic ccipabillties of the metabolite profile. To evaluate the potential of the metabolite profiles as a diagnostic panel, receiver operating characteristic curves were generated using partial least squares discriminant analysis models. FIG. 3 shows the area under the curve (AUC) values along with the 95% confidence intervals (CI) for four comparisons. Chart A of FIG. 3 evaluates the ability to discriminate between the control patients and the combined SV and SVHF group. Chart B of FIG. 3 shows the comparison between the control and SV group. The AUC and CI for this model are slightly better than far the combined group further suggesting that the SV and SVHF models are indeed metabolically distinct. The best performing ROC analysis was between the control and SVHF with the largest AUC value and the narrowest CI; chart C of FIG. 3. The AUG and CI comparing SV and SVHF (chart D of FIG. 3) illustrate that the metabolite panel possesses significant potential to discriminate SV patents from those with HF.

Pediatric SV patients are assessed to inform clinical decision making for children and young adults who are seen in the cardiology clinic. The metabolic biomarker panel is applied to SV patients to differentiate between patients in satisfactory, stable condition and patients who are in failure or progressing towards failure. Five SV patients with HF based on clinical examination were seen in the clinic. A metabolic biomarker panel was run on these patients according to embodiments. The biomarker panel detected heart failure with a high degree of accuracy, enabling the clinician to implement more aggressive management.

Application of embodiments provides for early detection of severe heart failure, enabling the patients to prepare for heart transplantation before their situation becomes moribund. Embodiments also inform clinical decision making in the SV patient such as diuretic management of HF and use of afterload reducing drugs to improve cardiac performance when the heart is failing, Diuretic drugs increase urination thus decreasing the amount of dilation (stretch) on the SV, whereas afterload reducing drugs decreases the work of the heart needed to pump blood out to the body. Embodiments permit comparison between the improvement (or not) of the heart function by comparing the patients' metabolomic profile across multiple time points. In addition, embodiments include the ability to plot the metaholomic profile reflecting the integrated body response of the patient to improved heart function by comparison against validated patterns for heart stable patients. These data and assays inform the clinician if selected drugs and medical management are effective, and, if not, the clinician may alter the patient's medical management protocol.

Further, the metabolic profiles found in this study significantly expand upon the limited metaholomic studies of SV subjects. These findings demonstrate that: 1) specific metabolic profiles are associated with HF in these patients, 2) these profiles can distinguish between Con, SV, and SVHF and, 3) the distinctions are found in early childhood. Multiple studies evaluating in the SV subject to this point have relied on clinical laboratory data, such as BNP, or multiplex immunoassays, and have advanced our understanding of inflammation and tissue responses at a protein level for the Fontan subject. However, the biochemical profiles of embodiments described herein provide new clues regarding the metabolic pathways that are altered in SV, and which patients may progress to HF.

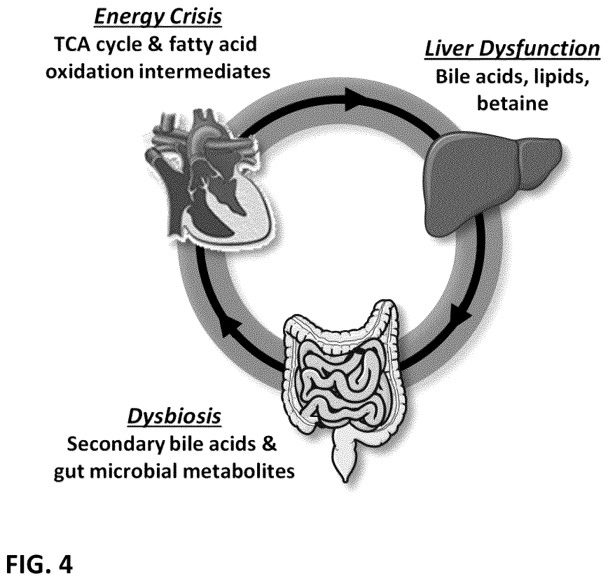

FIG. 4 provides an overview of how the various organs may be affected by the SV heart physiology, and how these metabolites may signal specific tissue dysfunction. This information was lacking in the field of congenital heart disease and will allow both clinical and basic interrogation of the Fontan physiology. A heart with a hypoplastic left ventricle is shown after an intermediate stage repair (Glenn shunt) prior to the Fontan operation. Here, the poor functioning of the heart can negatively affect downstream organs, such as liver and bowel. This altered physiology may, in turn, negatively affect cardiac function and the metabolites released by these organs may constitute a heart failure profile. With heart failure, the heart will release analytes from various metabolic activities, e.g., fatty acid oxidation, TCA cycle, glycolysis, etc. In addition, the poor perfusion in distal tissues, such as skeletal muscle and kidney, may result in release of analytes that can be measured in these assays. In the setting of heart failure, the liver will be poorly perfused and will function less well. Analytes expected from hepatic dysfunction would include bile acids, lipid dysregulation, betaine, and defects in protein synthesis. In addition, the liver in the. Single Ventricle patient is under much higher pressures than in a normal person and will have dysfunction in metabolism (glucose, fatty acid oxidation) as well as fibrosis. In heart failure, and especially in the Single Ventricle patient, the gut (small and large bowel) will have poor perfusion and decreased function. This allows growth of pathologic bacteria and decreases the microbial diversity in the gut (dysbiosis). These pathologic bacteria will release analytes and toxins into the blood stream that can be detected by NMR and Mass Spectrometry and provide information on the impact of poor perfusion and elevated pressure on the gut.

Recent studies invite a detailed comparison to the results presented here. Similar, albeit more limited, targeted metabolomics approaches were used in those studies and their cohort was similar in size to that described above. However, some differences are found in the subjects included in the earlier study when compared to those currently described. In the earlier studies, all of the SV subjects had a dominant left ventricle with 50% of the subjects having a double inlet left ventricle, and 50% having a variation of tricuspid atresia. In contrast, in in the present Examples, 50% of SV subjects had HLHS and thus, had dominant right ventricular anatomy. As has been noted by others, the outcome is not the same between right and left dominant SV hearts with significantly better function and survival of the LV dominant hearts. Secondly, subjects with failure of the systemic ventricle were excluded from tile earlier studies whereas the present Examples included them. Indeed, three of the subjects in the present Examples continued to transplant due to intractable HF. The present Examples also included subjects with protein losing enteropathy (PLE) whereas those subjects were excluded in the earlier study. Finally, the present Examples focused on children and young adults (age range 2.2 yr to 19.7 yr) whereas the earlier study included only adults and excluded subjects <18 yr of age. The results from the earlier study demonstrated that the metabolonne in adult SV patients can be distinguished from controls. The results from the present Examples, as described herein, further expand this distinction further and demonstrate that the metabolic phenotype of pediatric SV patients with heart failure is indeed distinguishable from pediatric SV patients with stable heart function.

A further distinction is that perturbations inn the acylcarnitine (AC) profiles were found in the earlier study of adult SV patients. In that study, significant elevations in C0 (carnitine), C3 (propionylcarnitine) and C18:2 (octadecadienylcarnitine) were thund in the SV patients. In contrast, perturbations were found in the present Examples only in dicarboxylated and hydroxylated species. Moreover, three of the four species were decreased while only one was increased, as shown in FIG. 2.

Other studies have found perturbations to ACs, and specifically, the dicarboxylated ACs. In a metabotomics analysis of serum from 314 individuals with CAD, a principal components analysis derived metabolite factor composed of small- to medium-chain dicarboxylated ACs significantly predicted an incidence of myocardial infarction (MI) and death. In a similar analysis from the CATHGEN cohort, a factor composed of both short- and long-chain dicarboxylated ACs was independently associated with mortality and MI. However, in the present Exarnples, the C12-DC and C16-OH metabolites were the only longer chain AC with a significant perturbation, but only in comparing the controls to SV and SVHF and not in the comparison of SV to SVHF. Changes in ACs in the present Examples as well as the studies of adults with CVD suggest that some perturbations to fatty acid metabolism in SV induced heart failure may be common with adult heart disease, while others may be distinct.

Amino acids may act as biosynthetic substrates for cellular structures and signaling molecules and as a source of energy. The circulating levels of amino acids are achieved in part by maintaining a balance between protein synthesis and degradation along with amino acid catabolism. Consistent with some earlier studies, decreases in Asp, His and Thr in the SV patients were found in the present Examples, and an increase in Glu in the SVHF compared with SV.

The alterations in BAs are an intriguing finding that may relate to the congestive hepatopathy associated with the Fontan circuit. Increases in serum BAs have been observed in a wide variety of liver diseases but not specifically described in the Fontan SV setting before the present disclosure. Interestingly, all 5 of the BAs were increased in the SVI-IF compared with controls, and three of these were different between SVHF and SV, suggesting they may be an early marker of hepatic pathology due to failure of the Fontan physiology. Bile acid metabolism is also affected by gut microbiota and is known to alter metabolism of the host. The significant increase in the secondary BA, GCA in the SVHF group compared to both controls and SV raises the possibility that the gut inicrobiome may contribute to or signal, HF in the SV patient. Further support for the role of gut microbiota is the increase in 3-indoleacetate, which is often produced as a product of gut microbial tryptophan metabolism.

A significant pattern of increased triglycerides was observed in the SV and SVHF group suggesting that dyslipidemia may be a factor in SV heart failure. Previous studies reported alterations in phospholipid metabolism as evidenced by reduced levels of phosphocholines and sphingomyelins. The increase in triglycerides found in the present Examples may be associated with the alteration in lipolytic processes that can play a significant role in the development of adult heart failures.

Previous studies have reported significant reductions in circulating cholesterol, including reductions in total cholesterol, low-density lipoprotein cholesterol, and high-density lipoprotein cholesterol in a cohort of pediatric SV patients who underwent Fontan palliation. In a subsequent study, evidence was found that the hypocholesterolemia was associated more with increased absorption rather than decreased synthesis. An observation, in this earlier study, of elevated markers of liver dysfunction suggested that the hypocholesterolemia was related to the abnormal hepatic cholesterol metabolism. Without intending to be bound by any particular theory, it is believed that the increased levels of TGs in the present Examples may be related to the decreased levels of cholesterol in earlier studies. It should be noted that both cholesterol and triglycerides are components of the low-density lipoprotein particles.

Other variations or embodiments will be apparent to a person of ordinary skill in the art from the above-description. Thus, the foregoing embodiments are not to be construed as limiting the scope of the claimed invention. All references disclosed are expressly incorporated by reference in in their entirety.

### REFERENCES

Hsu D T, Pearson G D. Heart failure in children: Part i: History, etiology, and pathophysiology. Circ Heart Fail. 2009; 2:63-70.

Ohye R G, Schranz D, D'Udekem Y. Current therapy for hypoplastic left heart syndrome and related single ventricle lesions, Circulation. 2016; 134:1265-1279.

d'Udekern Y, lyengar A J, Galati J C, Forsdick V, Weintraub R U, Wheaton G R, Bullock A, Justo R N, Grigg L E, Sholler G F, Hope S, Radford D J, Gentles Celermajer D S, Winlaw D S. Redefining expectations of long-term survival after the fontan procedure: Twenty-five years of follow-up from the entire population of Australia and New Zealand. Circulation. 2014; 130:532-38.

Emamaullee J, Zaidi A N, Schiano , Kahn J, Valentino P L, Hofer R E, Taper T, Wald J W, Olthoff K M, Bucuvalas J, Fischer R. Fontan-associated liver disease. Circulation. 2020; 142:591-604.

Reddy S, Handler S S, Wu S, Rabinovitch M. Wright G. Proceedings from the 2019 stanford single ventricle scientific summit: Advancing science for single ventricle patients: From discovery to clinical applications. J Am Heart Assoc. 2020; 9:e015871.

McGarrah R W, Crown S B, Zhang G E, Shah S H, Newgard C B. Cardiovascular inetabolomics. Ciro Res. 2018; 122:1238-1258.

Michel M, Dubowy K G, Entenmann A, Karall D, Adam M G, Zlamy M, Odri Komazee I, Geiger R, Niederwanger C, Salvador C, Muller U, Laser K T, Scholl-Burgi S. Targeted metabolomic analysis of serum amino acids in the adult fontan patient with a dominant left ventricle. Sci Rep. 2020; 10:8930.

Michel M, Dubowy K O, Zlamy M, Karall D, Adam M G, Entenmann A, Keller M A, Koch J, Odri Komazec I, Geiger R, Salvador C, Niederwanger C, Scholl-Burgi S, Laser K T. Targeted metabolomic analysis of serum phospholipid and acylcarnitine in the adult roman patient with a dominant left ventricle. Thee Adv Chronic Dis. 2020; 11:2040622320916031.

Beckonert O, Kenn H C, Ebbels T M, Bundy J, Holmes E, Lindon J C, Nicholson J K, Metabolic profiling, trietabolornic and inetabonomic procedures for nmr spectroscopy of urine, plasma, serum and tissue extracts. Nature protocols. 2007;2:2692-2703.

Cltoudhury R, Beezley , Davis B, Tomeck J, Gratzl S, Golzarri -Arroyo L, Wan J, Raftery D, Baumes J. O'Connell T M. Viime: Visualization and integration of metaboloinics experiments. The Journal of Open Source Software. 2020; 5:2410-2423.

Oster M E, Knight J H, Suthar D, Amin O, Kochilas L K. Long-term outcomes in single-ventricle congenital heart disease. Circulation. 2018; 138:2718-2720.

Erikssen G, Aboulhosn J, Lin J, Liestol K, Estensen M E, Gjesdal O, Skulstad H, Bohlen G, Lindberg H L. Survival in patients with univentricular hearts: The impact of right versus left ventricular morphology. Open Heart. 2018; 5:e000902.

Shah S R, Bain J R, Muehlbauer M J, Stevens R D, Crosslin D R, Haynes C, Dungan J, Newby L K, Hauser E R, Ginsburg G S, New-Mand C B, Kraus W E. Association of a peripheral blood metabolic profile with coronary artery disease and risk of subsequent cardiovascular events. Circ Cardiovasc. Genet. 2010; 3:207-214.

Hunter W G, Kelly J P, McGarrah R W, 3rd, Khouri M G, Craig D, Haynes. C. Ilkayeva O, Stevens R D, Bain J R, Muchlbauer M J, Newgard C B, Felker G M, Hernandez A F, Velazquez E J, Kraus W E, Shah S H, Metabolomic profiling identities novel circulating biomarkers of mitochondrial dysfunction differentially elevated in heart failure with preserved versus reduced ejection fraction: Evidence for shared metabolic impairments in clinical heart failure. J Am Heart Assoc, 2016; 5:e003190.

Neale G, Lewis B, Weaver V, Panveliwalla D. Serum bile acids in liver disease. Gut. 1971; 12:145-152.

Wahlstrom A, Sayin S I, Marschall H U, Backhed F. Intestinal crosstalk between bile acids and microbiota and its impact on host metabolism. Cell Metab. 2016; 24:41-50.

Kintseher U, Foryst-LudAvig A, Haemmerle G, Zechner R. The role of adipose triglyceride lipase and cytosolic lipolysis in cardiac function and heart failure. Cell Rep Med. 2020; 1:100001.

Ross R D, Medical management of chronic heart failure in children. Am J Cardiovasc Drugs, 2001; 137-44.

Connolly D, Rutkowski M, Auslender M, Artman M. The new York university pediatric heart failure index: A new method of quantifying chronic heart failure severity in children. J Pediatr. 2001; 138:644-648,

Tissieres P, Aggotin Y, Da Cruz E, Sierra J, Mensi N, Kalangos A, Beghetti M. Comparison of classifications for heart failure in children undergoing valvular surgery. J Pediatr. 2006; 149:210-215.

Atz, A. M., et al. (2017). Longitudinal Outcomes of Patients With Single Ventricle After the Fontan Procedure. J Am Coll Cardiol 69, 2735-2744 doi:10.1016/j.jacc.2017.03.581

Fredenhurg, T. B., T. R. Johnson, M. D. Cohen (2011). The Fontan procedure: anatomy, complications, and manifestations of failure. Radiographics 31, 453-63 doi:10.1148/rg.312105027.

Hoffman. J. I., S. Kaplan (2002). The incidence of congenital heart disease, J Am Coll Cardiol 39, 1890-900.

Pundi, K. N., et al. (2015). 40-Year Follow-Up After the Fontan Operation: Long-Term Outcomes of 1,052 Patients. J Am Coll Cardiol 66, 1700-10 doi:10.016/j.jacc.2015.07.065.

Reiter, M. D., M. J. Strickland, T. Riehle-Colarusso, W. T. Mable, A. Correa (2008). Prevalence of congenital heart detects in metropolitan Atlanta, 1998-2005. J Pediatr 153, 807-13 doi :10.1016/j.jpeds.2008.05.059,

van den Bosch, E., et al. (2021). Associations Between Blood Biomarkers, Cardiac Function, and Adverse Outcome in a Young Fontan Cohort. J Am Heart Assoc 10, e015022 doi:10.1161/JAHA.119.015022.

Whiteside, W., M. Tan, R. E. Ostlund, Jr., S. Yu, L Ma, A. Rocchini (2016). Altered Cholesterol Metabolism and Hypocholesterolernia in Patients with Single Ventricle following Fontan Palliation. J Pediatr 171, 73-7 doi:10.1016/jpeds.2015.12.038.

Whiteside, W., M. Tan, S. Yu, A. Rocehini (2013). Low total, low-density lipoprotein, high-density lipoprotein, and non-high-density lipoprotein cholesterol levels in patients with complex congenital heart disease after Fontan palliation. J Pediatr 162, 1199-204 doi:10.1016/j.jpeds.2012.11.073.

Zomer, A. C., et al. (2013). Heart failure admissions in adults with congenital heart disease; risk factors and prognosis. Int J Cardiol 168, 2487-93 doi:10.1016/j.ijcard.2013.03.003.

1. A method to assess a pediatric subject having a single ventricle heart, the method comprising:
determining a concentration level of one or more validated low molecular weight metabolic biomarkers in a biological sample from the subject;
identifying a difference between the determined concentration level of the one or more metabolic biomarkers and a reference concentration level of the one or more metabolic biomarkers; and,
assessing the pediatric subject based on the identified difference.

2. The method of claim 1, wherein the reference concentration level is determined from a healthy subject, a subject having single ventricle heart failure, or a combination thereof.

3. The method of claim 1, wherein assessing the pediatric subject is determining a stage in progression to heart failure.

4. The method of claim 3, wherein the stage is stable heart function.

5. The method of claim 3, wherein the stage is congenital heart failure.

6. The method of claim 1, wherein assessing the pediatric subject comprises predicting heart failure.

7. The method of claim 1, wherein the pediatric subject has been diagnosed as having heart failure and assessing the pediatric subject comprises determining a stage in progression of the heart failure.

8. The method of claim 1, wherein assessing the pediatric subject comprises determining a risk of developing heart failure.

9. (canceled)

10. The method of claim 1, further comprising:
calculating a ratio of the concentration levels of one or more validated low molecular weight metabolic biomarkers.

11. (canceled)

12. (canceled)

13. (canceled)

14. (canceled)

15. (canceled)

16. The method of claim 1, wherein the biological sample comprises a blood plasma sample.

17. The method of claim 1, wherein the one or more validated low molecular weight metabolic biomarkers comprises a panel of from five to ten validated low molecular weight metabolic biomarkers.

18. The method of claim 1, wherein the low molecular weight metabolic biomarker is selected from methylmalonylcarnitine, hydroxyvalerylcarnitine, pimeloylcarnitine, dodecanedioylcarnitine, hydroxyhexadecanoylcarnitine, aspartic acid, glutamic acid, histidine, 3-indolacetic acid, threonine, α-aminobutyric acid, γ-aminobutyric acid, nitro-tyrosine, ornithine, betaine, glycocholic acid, glycochenodeoxycholic acid, glycoursodeoxycholic acid taurocholic acid, taurochenodeoxycholic acid, dehydroepiandrosterone sulfate, hippurate, succinate citrate, FA(20.2), PC(32.0), DG(16.0:16.0), DG(18.1:18.1), DG(18.1:18.2), TG(16.0:36.3), TG(16.0:36.6), TG(18.0:36.2), TG(18.0:36.3), TG(18.1:36.1), TG(18.1:36.2), TG(18.1:36.3), TG(18.1:36.4), TG(18.1:36.5), TG(18.2:36.1), TG(18.2:36.2), TG(18.2:38.4), TG(18.2:38.5), TG(20.5:34.1), TG(20.5:34.2), TG(20.5:36.3), or a combination of two or more thereof.

19. (canceled)

20. The method of claim 1, wherein the pediatric subject is undergoing a treatment for single ventricle heart and the treatment is continued, discontinued, or altered based on presence, absence, or the concentration level of the one or more low molecular weight metabolic biomarkers.

21. The method of claim 1, further comprising:
providing a recommended treatment based on presence, absence, or concentration level of the one or more low molecular weight metabolic biomarkers; and
administering the treatment to the pediatric subject.

22. The method of claim 1, wherein the low molecular weight metabolic biomarker comprises an acylcarnitine.

23. (canceled)

24. (canceled)

25. The method of claim 1, wherein the low molecular weight metabolic biomarker comprises an amino acid.

26. (canceled)

27. (canceled)

28. The method of claim 1, wherein the low molecular weight metabolic biomarker comprises a bile acid.

29. (canceled)

30. (canceled)

31. The method of claim 1, wherein the low molecular weight metabolic biomarker comprises a lipid.

32. (canceled)

33. (canceled)

34. The method of claim 1, wherein the low molecular weight metabolic biomarker comprises a steroid.

35. (canceled)

36. (canceled)

37. The method of claim 1, wherein the low molecular weight metabolic biomarker comprises a product of benzoate metabolism.

38. (canceled)

39. The method of claim 1, wherein the low molecular weight metabolic biomarker comprises a product of aromatic amino acid synthesis.

40. (canceled)

41. The method of claim 1, wherein the low molecular weight metabolic biomarker comprises a Krebs cycle intermediate.

42. (canceled)

43. (canceled)

44. The method of claim 1, wherein the concentration level of the one or more low molecular weight metabolic biomarkers in the biological sample is determined by a chromatography and/or spectrometry method.

45. (canceled)

46. (canceled)

47. The method of claim 1, further comprising:
altering a treatment course of action.

48. (canceled)

49. A diagnostic kit for assessing an attribute of heart failure in a pediatric subject having a single ventricle heart disease, where the attribute of heart failure is associated with a low molecular weight metabolic biomarker profile, the kit comprising:
a container comprising
at least one validated low molecular weight metabolic biomarker internal standard having a purity greater than 98.0%,
a sample receiving vessel for receiving a biological sample, and
a sealing member for sealing the sample receiving vessel after receiving the biological sample.

50. The diagnostic kit of claim 49, wherein the pediatric subject is an infant human subject.

51. (canceled)

52. (canceled)

53. The diagnostic kit of claim 49, wherein the biological sample comprises a blood plasma sample.

54. The diagnostic kit of claim 49, wherein the one or more validated low molecular weight metabolic biomarkers comprises a panel of from five to ten validated low molecular weight metabolic biomarkers.

55. The diagnostic kit of claim 49, wherein the low molecular weight metabolic biomarker is selected from methylmalonylcarnitine, hydroxyvalerylcarnitine, pimeloylcarnitine, dodecanedioylcarnitine, hydroxyhexadecanoylcarnitine, aspartic acid, glutamic acid, histidine, 3-indolacetic acid, threonine, α-aminobutyric acid, γ-aminobutyric acid, nitro-tyrosine, ornithine, betaine, glycocholic acid, glycochenodeoxycholic acid, glycoursodeoxycholic acid taurocholic acid, taurochenodeoxycholic acid, dehydroepiandrosterone sulfate, hippurate, succinate citrate, FA(20.2), PC(32.0), DG(16.0:16.0), DG(18.1:18.1), DG(18.1:18.2), TG(16.0:36.3), TG(16.0:36.6), TG(18.0:36.2), TG(18.0:36.3), TG(18.1:36.1), TG(18.1:36.2), TG(18.1:36.3), TG(18.1:36.4), TG(18.1:36.5), TG(18.2:36.1), TG(18.2:36.2), TG(18.2:38.4), TG(18.2:38.5), TG(20.5:34.1), TG(20.5:34.2), TG(20.5:36.3), ora combination of two or more thereof.

56. The diagnostic kit of claim 49, wherein the low molecular weight metabolic biomarker comprises an acylcarnitine.

57. (canceled)

58. (canceled)

59. The diagnostic kit of claim 49, wherein the low molecular weight metabolic biomarker comprises an amino acid.

60. (canceled)

61. (canceled)

62. The diagnostic kit of claim 49, wherein the low molecular weight metabolic biomarker comprises a bile acid.

63. (canceled)

64. (canceled)

65. The diagnostic kit of claim 49, wherein the low molecular weight metabolic biomarker comprises a lipid.

66. (canceled)

67. (canceled)

68. The diagnostic kit of claim 49, wherein the low molecular weight metabolic biomarker comprises a steroid.

69. (canceled)

70. (canceled)

71. The diagnostic kit of claim 49, wherein the low molecular weight metabolic biomarker comprises a product of benzoate metabolism.

72. (canceled)

73. The diagnostic kit of claim 49, wherein the low molecular weight metabolic biomarker comprises a product of aromatic amino acid synthesis.

74. (canceled)

75. The diagnostic kit of claim 49, wherein the low molecular weight metabolic biomarker comprises a Krebs cycle intermediate.

76. (canceled)

77. (canceled)

78. A method to identify a candidate low molecular weight metabolic biomarker that differentiates a pediatric subject having a single ventricle heart without heart failure and a pediatric subject that has a single ventricle heart with heart failure, the method comprising:
obtaining a first biological sample from a pediatric subject having a single ventricle heart without heart failure and a second biological sample from a pediatric subject having a single ventricle heart with heart failure;
determining a concentration level of one or more low molecular weight metabolic biomarkers in the first biological sample and the second biological sample; and,
identifying one or more validated low molecular weight metabolic biomarkers that are present in a statistically significant, different level in the first and second biological samples, where the different level is characterized by an area under the receiver operator characteristic (ROC) curve (AUC) ranging from 0.60 to 1.00.
